(19) United States
(12) Patent Application Publication
(10) Pub. No.: US 2019/0272960 A1
(43) Pub. Date: Sep. 5, 2019

(54) DOPANT-FREE INEXPENSIVE HOLE TRANSPORTING MATERIALS FOR HIGHLY EFFICIENT AND STABLE PEROVSKITE SOLAR CELLS

(71) Applicant: Curators of the University of Missouri, Columbia, MO (US)

(72) Inventors: Zhonghua Peng, Overland Park, MO (US); Yong Li, Kansas City, MO (US); Kathleen Kilway, Kansas City, MO (US)

(21) Appl. No.: 16/345,537

(22) PCT Filed: Oct. 27, 2016
(86) PCT No.: PCT/US2017/058334

§ 371 Date: Apr. 26, 2019

Related U.S. Application Data
(60) Provisional application No. 62/413,755, filed on Oct. 27, 2016.

(51) Int. Cl. H01G 9/20; H01L 51/42; H01L 51/44; C07D 333/78; H01L 51/00

(52) U.S. Cl. CPC H01G 9/2009; H01L 2251/301; H01G 9/2027; H01G 9/2031; H01G 9/204; H01L 51/422; H01L 51/4226; H01L 51/4233; H01L 51/442; C07D 333/78; H01L 51/0077; H01L 51/0074; H01L 2251/306; H01L 2251/308; H01G 9/2018

(57) ABSTRACT

Disclosed herein are novel hole transporting materials comprising polycyclic heteroaromatic hydrocarbon compounds that are easy to synthesize and inexpensive. The hole transporting materials of the present disclosure have high hole mobility and thus do not require any doping. The hole transporting materials of the present disclosure also have matching frontier orbitals when used in devices with the perovskite and cathodes, facilitating hole migration across perovskite/hole transporting layers and hole transporting layer/cathode interfaces. The hole transporting materials of the present disclosure can further be hydrophobic with no moisture attracting atoms. The hole transporting materials can be used to form dense and uniform films on perovskite, and combined with hydrophobicity, form an excellent moisture barrier for the perovskite. With the present disclosure compound as the hole transporting layer in a perovskite solar cell, highly stable and highly efficient and inexpensive solar cells can be achieved.

### CROSS REFERENCE TO RELATED APPLICATIONS

This application claims priority to U.S. Ser. No. 62/413,755, filed on Oct. 27, 2016, which is incorporated by reference herein in its entirety.

### STATEMENT OF GOVERNMENT SUPPORT

This invention was made with government support under DMR1308577 awarded by the National Science Foundation. The Government has certain rights in the invention.

### BACKGROUND OF THE DISCLOSURE

The present disclosure is generally directed to the application of a type of substituted polycyclic heteroaromatic compounds as the hole transporting material in perovskite solar cells. More particularly, the present disclosure is directed to thin films including compounds having polycyclic heteroaromatic compounds. The present disclosure is further directed to the applications of such compounds in solar cells, light emitting diodes, and transistors.

Perovskite solar cells (PSCs) have emerged as an appealing technology for solar-electricity conversion. Perovskite solar cells were first reported in 2009 with a power conversion efficiency lower than 4%. In just a few years, the efficiency of perovskite solar cells has skyrocketed to over 22%, surpassing multicrystalline Si-based solar cells and making it undoubtedly the most appealing new technology for solar-electricity conversion. In addition to higher efficiencies, perovskite solar cells, compared to Si-based solar cells, have other advantages such as simple and easy solution fabrication processes, flexible substrates, lower weight, and amenable to a variety of lighting conditions. It is thus the consensus of the scientific community that perovskite solar cells will significantly impact if not dominate the solar cell market.

Perovskite solar cells have a perovskite-structured compound as the light absorbing layer. The perovskite layer is most commonly a hybrid organic-inorganic lead halide of molecular formula ABX₃, where A is an organic cation, typically methylammonium cation or formamidinium cation, or a combination of both and sometimes with Cs⁺, B is Pb²⁺ or Sn²⁺ and X is a halide or a combination of two halides.

When light is absorbed by a perovskite, a bound electron-hole pair (called an exciton) with a bonding energy as low as 25 meV is produced, which can easily split into free charge carriers under a small bias. The generated free electrons move towards and eventually are collected by the anode while holes migrate towards and are collected by the cathode. To facilitate electron and hole collection and minimize their combination, an interfacial layer between the electrode and the perovskite is needed. The layer between the perovskite and the anode is called the electron transporting layer (ETL), while the interfacial layer between perovskite and the cathode is called the hole transporting layer (HTL). A perovskite solar cell thus typically has an anode/ETL/Perovskite/HTL/cathode device configuration.

Depending on which electrode is attached to the transparent substrate, two major device architectures have been investigated. An n-i-p structure has the anode on the substrate with a device configuration of substrate/anode/ETL/perovskite/HTL/cathode, while the p-i-n structure has the cathode on the substrate with a device configuration of substrate/cathode/HTL/perovskite/ETL/anode. The anode is often a conductive oxide, typically indium-tin-oxide (ITO), or fluorine-doped tin oxide (FTO). The cathode is often a high work function metal such as gold or silver. The ETL can be an organic compound, a polymer, or n-type (electron transporting) oxide such as TiO₂ or ZnO. When oxides are used, they can either be a single compact blocking layer or a double layer structure with a mesoporous perovskite-infiltrated scaffold on top of the compact blocking layer. The later device configuration is often called meso-structured perovskite solar cells (MSSC).

HTL plays a critical role in device performance. The ideal HTL materials should have matching frontier orbital levels with both perovskite and the cathode, but also high hole transporting mobility and excellent moisture-resistant properties. Despite advances in the development of HTL materials, 2,2′,7,7′-tetrakis(N,N-di-p-methoxy-phenylamine)-9,9′-spirobifluorene (spiro-OMeTAD) remains the HTL of choice with the best reported performance Spiro-OMeTAD is currently prohibitively expensive, however. Spiro-OMeTAD is also a poor conductor, and thus, requires a complicated doping process that is difficult to control. The dopants are often ions that attract moisture which degrades the perovskite layer. Realizing that perovskite solar cells with spiro-OMeTAD as the HTL is not going to be commercially viable, there have been tremendous research efforts devoted towards developing new HTL materials. However, all top performing HTL materials reported so far have complicated structures involving multi-step syntheses and thus are expensive. Most of them also do not have sufficient hole mobility and thus still require doping. All of them contain multiple N and/or O atoms which attract moisture and thus are detrimental to device stability.

Accordingly, there is an urgent need for alternative dopant-free hole extraction/transporting layer (HTL) materials that are inexpensive and produce perovskite solar cells with high efficiency and high stability.

### SUMMARY OF THE DISCLOSURE

In view of the foregoing, the present disclosure provides novel hole transporting materials that are easy to synthesize and inexpensive, costing only about a hundredth of the price of spiro-OMeTAD. The hole transporting materials of the present disclosure also have high hole mobility and thus do not require any doping. The hole transporting materials of the present disclosure have matching frontier orbitals with the perovskite and the cathode, facilitating hole migration across perovskite/HTL and HTL/cathode interfaces. The hole transporting materials of the present disclosure are hydrophobic with no moisture attracting oxygen and/or nitrogen atoms. The hole transporting materials of the present disclosure can form dense and uniform film on top of perovskite, and combined with hydrophobicity, forming an excellent moisture barrier for the perovskite underneath. Using the disclosed compounds as the HTL material, perovskite solar cells with high power conversion efficiencies and high long-term stability can be realized.

The present disclosure is generally directed to the application of a type of substituted polycyclic heteroaromatic compounds as the hole transporting material in perovskite solar cells. More particularly, the present disclosure is directed to thin films including compounds having polycyclic heteroaromatic compounds. The present disclosure is further directed to the applications of such compounds in solar cells, light emitting diodes, and transistors.

In one aspect, the present disclosure is directed to a thin film composition comprising a compound of formula (I)

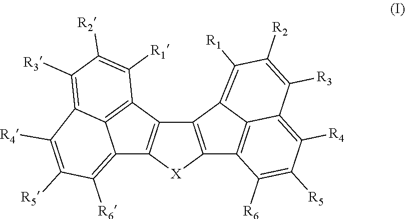

wherein X is a heteroatom of O, S, Se, and N—R′; and R₁, R₂, R₃, R₄, R₅, R₆, R₁′, R₂′, R₃′, R₄′, R₅′, R₆′, and R′, independently comprise a solubilizing group.

In one aspect, the present disclosure is directed to a perovskite photovoltaic device comprising: a perovskite layer and a hole transporting layer, wherein the hole transporting layer comprises a polycyclic heteroaromatic hydrocarbon.

### DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 depicts the chemical structure of one exemplary hole transporting compound (PCA-1).

FIGS. 2A & B depict the differential scanning calorimetry thermograms, showing its high temperature stability.

FIG. 3 depicts the X-ray diffraction patterns of the thin film of the exemplary compound before and after thermal annealing at 120° C. for 10 min, showing the high crystallinity of the thin film after annealing.

FIG. 4 depicts experimental (circles) and fitted (lines) current density-voltage (J-V) characteristics of the hole-only devices of thin films of PCA-1 with the configuration of ITO/PEDOT:PSS/PCA-1/MoO₃/Au with and without thermal annealing.

FIGS. 5A & 5B depict device architectures used to investigate an exemplary compound as the hole-transporting layer in perovskite solar cells.

FIGS. 6A & 6B depict cross sectional (FIG. 6A) and surface FESEM images (FIG. 6B) of the planar n-i-p perovskite device, showing the dense, uniform and pinhole-free film formed by the exemplary hole transporting compound.

FIG. 7 depicts current density-voltage (J-V) curves of perovskite solar cells using an exemplary compound as the hole-transporting layer in device architectures FIGS. 5A and 5B.

FIG. 8 depicts the current density-voltage (J-V) curves of perovskite solar cells using an exemplary compound as the hole-transporting layer in device architectures 5A and 5B after storing the devices at ambient conditions for an extended period of time.

FIG. 9 is a schematic depicting the synthesis of 2,5,9,12-tetra-tert-butyldiacenaphtho[1,2-b:1′,2′-d]thiophene (PCA-1) from acenaphthene.

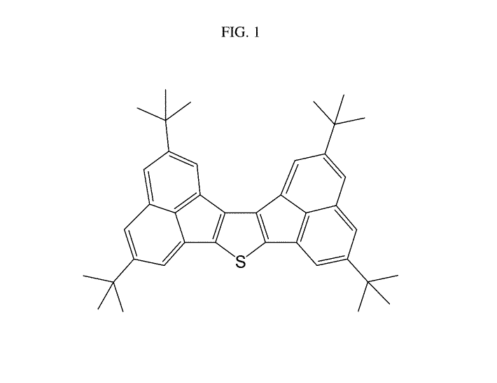

### DETAILED DESCRIPTION

Disclosed herein are polycyclic heteroaromatic compounds that provide excellent HTL materials for perovskite solar cells. These compounds are easy to synthesize and inexpensive. These compounds advantageously, provide one of the fastest hole-transporting mobilities of any organic compounds. These compounds further are hydrophobic with excellent moisture resistance. These compounds also exhibit the desired matching frontier orbitals. When used as the HTL without any dopants, the PSCs show significantly improved performance in terms of both efficiency and device stability over PSCs with spiro-OMeTAD as the HTL.

As used herein, “hole” refers to a positively charged carrier. While “electron” is a negative charge-carrier. A “p-type semiconductor” refers to semiconductors with holes as the majority charge carriers while an “n-type semiconductor” has electrons as the majority charge carriers.

As used herein, “mobility” of a charge carrier refers to the velocity of the charge carrier moving through the material under an electric field. The hole (or electron) mobility of a thin film can be measured using space-charge limited current method (SCLC) on a hole-only (or electron-only) diode device.

As used herein, short circuit current density (Jsc) refers to the current density of a solar cell at zero voltage across the solar cell. Open circuit voltage (Voc) refers to the difference of electrical potential between two electrodes of a device when there is no external load connected.

As used herein, the “fill factor” (FF) of a solar cell refers to the maximum obtainable power (Jₘₚ*Vₘₚ) to the theoretical power (J_{sc}*V_{oc}). Jₘₚ and Vₘₚ are the current density and voltage, respectively, at the maximum power point. In other words, FF=(Jₘₚ*Vₘₚ)/(J_{sc}V_{oc}).

As used herein, the power conversion efficiency of a solar cell refers to the percentage of power converted from the illuminating solar energy to electrical energy. The power conversion efficiency of a solar cell can be calculated by dividing the maximum power by the input light irradiance.

The term “hydrophobic” is used herein according to its ordinary meaning to refer to a physical property of a molecule that repels (or not attract) water. Such molecules often have non-polar bonds.

The terms “halo” or “halides” are used herein according to their ordinary meaning to refer to fluoro, chloro, bromo, and iodo.

The term “alkyl” is used herein according to its ordinary meaning to refer to a straight or branched hydrocarbon with only C—C and C—H single bonds.

The term “cycloalkyl” is used herein according to its ordinary meaning to refer to a carbocyclic group that has only single bonds. Common cycloalkyls include cyclohexyl, cyclopentyl groups.

The term “haloalkyl” is used herein according to its ordinary meaning to refer to an alkyl group with one or more Hs replaced by a halogen. Examples of haloalkyl groups include CHF₂, CH₂F, CF₃, CCl₃, and the like.

The term “alkenyl” is used herein according to its ordinary meaning to refer to a hydrocarbon chain, straight or branched, containing one or more carbon-carbon double bonds. The double bond can be terminal or internal.

The term “alkynyl” is used herein according to its ordinary meaning to refer to a hydrocarbon chain, straight or branched, containing one or more carbon-carbon triple bonds. The triple bond can be terminal or internal.

The term “aryl” is used herein according to its ordinary meaning to refer to a conjugated, cyclic or polycyclic system that is aromatic. Aromatic refers to the special stability of II-electrons in some cyclic conjugated II-systems.

In one aspect, the present disclosure is directed to a hole transporting compound of formula (I)

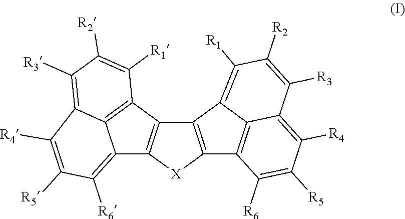

wherein X independently is a heteroatom of O, S, Se, and N—R′; R₁, R₂, R₃, R₄, R₅, R₆, R₁′, R₂′, R₃′, R₄′, R₅′, R₆′, and R′ independently is a solubilizing group.

Suitable R₁, R₂, R₃, R₄, R₅, R₆, R₁′, R₂′, R₃′, R₄′, R₅′, R₆′, and R′ solubilizing groups include alkyl, cycloalkyl, haloalkyl, alkenyl, alkynyl and arylalkyl groups. Particularly suitable R₁, R₂, R₃, R₄, R₅, R₆, R₁′, R₂′, R₃′, R₄′, R₅′, R₆′, and R′ can, at each occurrence, independently be H or a C₁₋₂₀ alkyl group, or a C₅₋₁₄ cycloalkyl group, or a C₁₋₂₀ haloalkyl group, or a —Ar—R″ group, where R″ is a solubilizing group; Ar, at each occurrence, independently is a C₆₋₁₄ aryl group. Preferably, R₁, R₃, R₄, R₆, R₁′, R₃′, R₄′, and R₆′ are H, while R₂, R₅, R₂′ and R₅′ are alkyl, alkenyl, or haloalkyl groups. Attaching alkyl chains (or similar groups such as alkenyl groups, alkynyl groups, haloalkyl groups, arylalkyl groups, and so forth) to the periphery of the polycyclic heteroaromatic core can improve its solubility in various organic solvents and can also impact the packing of the compounds in thin films. Particularly suitable R₂, R₅, R₂′ and R₅′ include a C₁₋₂₀ alkyl group, a C₁₋₂₀ haloalkyl group, and a —Ar—R″, wherein Ar is a C₆₋₁₄ aryl group and R″ is a C₁₋₂₀ alkyl or haloalkyl group.

In one embodiment, the polycyclic aromatic hydrocarbon is a compound of formula (II)

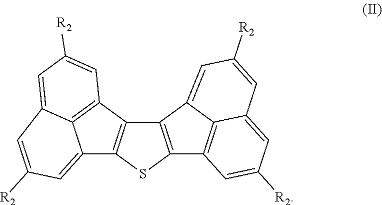

Suitably, each R₂ can be a C₁₋₂₀ alkyl group, a C₁₋₂₀ haloalkyl group, and a —Ar—R″, wherein Ar is a C₆₋₁₄ aryl group and R″ is a C₁₋₂₀ alkyl or haloalkyl group.

In another aspect, the present disclosure is directed to methods of synthesizing hole transporting compounds. Hole transporting compounds can be prepared following procedures analogous to those described in the examples. In particular, the diacenaphtho[1,2-b:1′,2′-d]thiophene core can be prepared in one simple step from commercially available inexpensive acenaphthylene with sulfur. The peripheral solubilizing R groups can be introduced into acenaphthylene before its reaction with sulfur or after the diacenaphtho[1,2-b:1′,2′-d]thiophene core formation using standard synthetic methods and procedures known to those in the art. Those skilled in the art of organic synthesis will recognize that the nature and order of the synthetic steps presented can be varied for forming the compounds described herein.

The method can further include purification of the compounds. Compounds can be purified using standard techniques. The purity of the compounds can be confirmed by methods such as elemental analysis, mass spectrometry, nuclear magnetic resonance spectroscopy (NMR, ¹H or ¹³C).

The compounds are stable under ambient conditions and at high temperatures (about 400° C.), which permits their use in devices intended to be operated in harsh environmentally-demanding conditions such as high humidity and high temperature.

The compounds can be soluble in various common organic solvents. As used herein, a compound is considered soluble in a solvent when at least 1 mg of the compound can be dissolved in 1 mL of the solvent. Suitable organic solvents include aliphatic and aromatic hydrocarbons such as hexane, benzene, toluene; halogenated aliphatic and aromatic hydrocarbons such as chloroform, dichlorobenzene; alcohols such as methanol, ethanol; ethers such as diethylether, tetrahydrofuran (THF); ketones such as acetone; esters such as ethyl acetate; amides such as dimethylformamide (DMF); and sulfoxides such as dimethylsulfoxide (DMSO).

The hole transporting compounds of the present disclosure are particularly suitable for use in thin films. Thus, in another aspect, the present disclosure is directed to a thin film including the compound of formula (I). Thin films can be fabricated using solution processing techniques as well as other more expensive processes such as high vacuum vapor deposition. A number of solution processing techniques have been used to fabricate organic molecular electronics. These techniques include, for example, spin coating, drop casting, dip coating, blade coating, spraying, and printing. As used herein, a thin film is one having a thickness less than about 5 μm. Particularly suitable thin film thickness can range from about 10 nm to about 5 μm, including ranging from about 10 nm to 1000 nm. The thickness of a thin film can be measured using techniques such as profilometry, ellipsometry, and spectrophotometric measurements.

Thin films can be pin-hole free, crack-free, and uniform. Thin films can further be hydrophobic, preventing moisture infiltration through the film.

Thin films of the present compounds can exhibit high charge carrier mobility with or without thermal annealing. The high mobility compounds disclosed herein can be used in photovoltaic devices, but also in a number of other optical, optoelectronic and electronic devices such as conductivity-based sensors, organic field-effect transistors (OFETs), light-emitting diodes (LEDs), and organic lasers. The compounds disclosed herein offer advantages in low cost, easy solution processing, high stability, high charge mobility and good device performance.

In another aspect, the present disclosure is directed to a perovskite photovoltaic device including a perovskite layer and a hole transporting layer, wherein the hole transporting layer comprises a polycyclic heteroaromatic hydrocarbon compound, as disclosed herein. A particularly suitable polycyclic heteroaromatic hydrocarbon includes a compound with a diacenaphtho[1,2-b:1′,2′-d]thiophene, as disclosed herein. In one embodiment, the perovskite device includes at least one electrode, a hole transporting layer, and a perovskite layer. In one embodiment, the perovskite device can further include an electron transporting layer (ETL) between the perovskite and the at least one electrode. The at least one electrode can be an anode, wherein the anode includes a transparent conducting oxide such as indium tin oxide (ITO) and fluorine-doped tin oxide (FTO). The at least one electrode can be a metal cathode, wherein the metal includes gold, silver, and aluminum. In some embodiments, the cathode is a C-electrode.

In some embodiments, the perovskite photovoltaic device can have a planar device structure. In certain embodiments, the planar device structure can have the device configuration of substrate/anode/ETL/perovskite/HTL/cathode, which is called the n-i-p device structure. In other embodiments, the planar device can have an inverted structure with a device configuration of substrate/cathode/HTL/perovskite/ETL/anode, which is called the p-i-n structure. Suitably, the anode can be a conductive oxide. Suitable conductive oxides include indium-tin-oxide (ITO) and fluorine-doped tin oxide (FTO). Suitably, the cathode can be a high work function metal such as gold and silver. The ETL can be an organic compound, a polymer, or n-type (electron transporting) oxide such as TiO₂ and ZnO. The HTL is a hole-transporting material described herein.

In some embodiments, the perovskite photovoltaic device can have an interfacial layer between the at least one electrode and the perovskite layer. The interfacial layer can suitably include TiO₂, ZnO, and Al₂O₃.

In some embodiments, the perovskite photovoltaic device can have a mesoporous metal oxide layer infiltrated with perovskite between the ETL layer and the perovskite layer with a device configuration of substrate/anode/ETL/mesoporous+perovskite/perovskite/HTL/cathode. Such devices are called mesostructured perovskite solar cells (MSSCs). Some of the metal oxides used for mesoporous scaffold include TiO₂, ZnO, Al₂O₃, and ZrO₂.

The perovskite can have a perovskite structure with a composition formula of ABX₃, where A is an organic cation, B is Sn²⁺ and Pb²⁺, and X is a halide. Suitable organic cations include CH₃NH₃⁺ (methylammonium or MA), HN═CHNH₃⁺ (formamidinium or FA), (CH₂)₃NH₂⁺ (azetidinium), and [C(NH₂)₃]⁺ (guanidinium). Methylammonium lead triiodide (MAPbI₃) is so far the most extensively studied and one of the most efficient materials for solar cells. In some embodiments, a combination of cations and halides can be used. For example, perovskites of MAₓFA₁₋ₓPbI_{y}Br_{3-y} where x is 0-1 and Y is 0-3 can be used. Varying x and y can affect the device efficiency and stability. In some embodiments, multivalent organic cations such as ethane-1,2-diammonium or inorganic cations such as Cs⁺ can also be mixed with monovalent organic cations. The perovskite layer can have a thickness ranging from about 50 nm to about 800 nm.

The perovskite has a perovskite crystalline structure. The size, shape, and structure of the perovskite crystals have profound effect on the device performance Many deposition techniques and protocols have been developed to optimize the resulting perovskite structure. Solution-based deposition methods include spin-coating, spraying, and ink-jet printing. The source (precursor) materials for perovskites are often metal halides (e.g. PbI₂) and organic cation halide (e.g., methylammonium iodide (MAI) and formamidinium iodide (FAI)). The two precursors can be deposited together in one solution, or sequentially in two separate solutions. After deposition, thermal annealing can be performed to remove solvent and to form the perovskite crystalline structure.

Suitable substrate materials of the perovskite solar cell are known in the art and can include rigid and hard materials and flexible and soft materials. Suitable substrate materials include glass, plastics, and elastomeric films, for example. Particularly suitable substrate materials include transparent substrates.

In one embodiment, the hole transporting layer is substantially free of a dopant. As used herein, the hole transporting layer is “substantially free of a dopant” when the amount of dopant less than 0.1% dopant, more particularly less than 0.01% dopant, and more suitably includes 0.0% dopant.

Suitable polycyclic heteroaromatic hydrocarbon compounds include diacenaphtho[1,2-b:1,2′-d]thiophene, diacenaphtho[1,2-b:1,2′-d]senelophene, diacenaphtho[1,2-b:1′,2′-d]furan, and diacenaphtho[1,2-b:1′,2′-d]pyrrole. A particularly suitable polycyclic aromatic hydrocarbon compound includes 2,5,9,12-tetra(tert-butyl)diacenaphtho[1,2-b:1′,2′-d]thiophene.

Suitably, the hole transporting layer has a space charge limited current hole mobility ranging from about 10⁻⁴ cm² V⁻¹ s⁻¹ to about 10⁻¹ cm² V⁻¹s⁻¹.

Suitably, the hole transporting compound has a highest occupied molecular orbital energy level of about −5.40 eV.

The hole transporting layer can range from about 10 nm to about 1000 nm.

The polycyclic heteroaromatic compounds disclosed herein provide excellent HTL materials for perovskite solar cells.

As described herein and in the Examples below, the present compounds without any doping, when used as the hole transporting layer in perovskite solar cells can lead to exceptionally high power conversion efficiencies (PCE) over 15.5% (up to 17.81%) and high stability. As far as the inventor's knowledge, this is the highest efficiency ever achieved on a similar dopant-free device using the same perovskite material and measured the performance of the whole physical device without blocking (eliminating) any edge effects.

### EXAMPLES

### Example 1

In this Example, the synthesis of 2,5,9,12-tetra-tert-butyldiacenaphtho[1,2-b:1′,2′-d]thiophene is described.

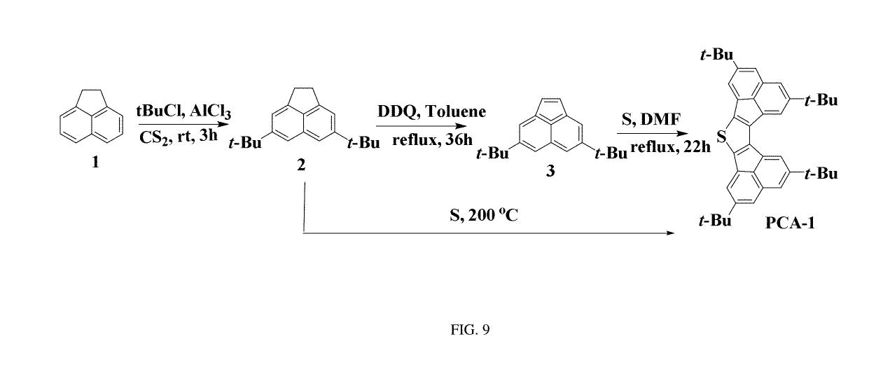

Referring to FIG. 9, 2,5,9,12-tetra-tert-butyldiacenaphtho[1,2-b:1′,2′-d]thiophene (PCA-1) was synthesized in three steps from commercially available acenaphthene (1). Alkylation of (1) under Friedel-Crafts conditions gave 4,7-di-tert-butyl-1,2-dihydroacenaphthylene (2) in 28% yield. Dehydrogenation of (2) with 2,3-dichloro-5,6-dicyano-1,4-benzoquinone (DDQ) in refluxing toluene produced 4,7-di-tert-butylacenaphthylene (3) in 64% yield. Finally, compound (3) was refluxed with sulfur in N,N-dimethylformamide (DMF) for 22 hours to give PCA-1 in 74% yield, with an overall yield of 13%. The reaction can be scaled up and multigram quantities of the product PCA-1 have been successfully synthesized in one batch in an academic lab setting. PCA-1 can also be synthesized in one step from (2) by heating with sulfur for several hours at over 200° C. The reaction was lower yield (<10%) and the isolation of the pure product from the reaction mixture was difficult.

To synthesize 4,7-di-tert-butyl-1,2-dihydroacenaphthylene (2), tert-butylchloride (20 mL, 180 mmol) was added dropwise to a suspension of acenaphthene (15.1 g, 97.9 mmol) and AlCl₃ (2.65 g, 19.9 mmol) in carbon disulfide (100 mL) over 25 minutes. The reaction contents were stirred for 3 hours at room temperature and then refluxed for 1 hour. Water (100 mL) was cautiously added, followed by concentrated HCl (5 mL). The mixture was extracted with CS₂, dried over Na₂SO₄, and filtered. The solvent was removed under reduced pressure. Recrystallization from acetic acid, followed by recrystallization from toluene/EtOH gave 4,7-di-tert-butyl-1,2-dihydroacenaphthylene (2) as a colorless solid (7.42 g, 27.9 mmol, 28.4%): mp 161-162° C.; ¹H NMR (400 MHz, CDCl₃) δ 1.43 (s, 18H), 3.40 (s, 4H), 7.36 (t, J=0.5 Hz, 2H), 7.55 (d, J=1.0 Hz, 2H); ¹³C NMR (100 MHz, CDCl₃) δ 30.5, 31.7, 35.3, 117.4, 130.7, 136.3, 145.1, 151.2; HRMS (APPI-TOF) calculated for C₂₀H₂₆ 266.2029, found 266.2040.

To synthesize 4,7-di-tert-butylacenaphthylene (3), acenaphthene (2) (10.1 g, 37.9 mmol) and DDQ (8.59 g, 37.8 mmol) were refluxed in toluene (250 mL) for 36 hours. The reaction contents were vacuum filtered, and the solid rinsed with toluene. The filtrate was washed with 10% NaOH (3×150 mL) and saturated NaCl (150 mL). The organic layer was dried over magnesium sulfate and filtered. The solvent was removed under reduced pressure. The residue was subjected to column chromatography (silica gel; solvent:hexanes). A yellow band was collected Rf 0.46 TLC (silica gel; solvent:hexanes). Recrystallization from ethanol gave 4,7-di-tert-butylacenaphthylene (3) as a yellow solid (6.43 g, 24.3 mmol, 64.1%): mp 108.5-109.5° C.; ¹H NMR (400 MHz, CDCl₃) δ 1.58 (s, 18H), 7.20 (s, 2H), 7.89 (d, J=1.0 Hz, 2H), 7.91 (d, J=1.0 Hz, 2H); ¹³C NMR (100 MHz, CDCl₃) δ 31.7, 35.5, 122.0, 122.6, 125.4, 127.2, 129.6, 139.0, 151.1; HRMS (APPI-TOF) calculated for C₂₀H₂₄ 264.1873, found 264.1881.

To synthesize 2,5,9,12-tetra-tert-butyldiacenaphtho[1,2-b:1′,2′-d]thiophene (PCA-1), 4,7-di-tert-butylacenaphthylene (8.45 g, 32.0 mmol) and sulfur (Calcd. as S₈, 4.15 g, 16.2 mmol) were refluxed in DMF (130 mL) for 22 h. The reaction contents were cooled to r.t. and vacuum filtered. The solid was washed with methanol to give the product as an orange solid, which was recrystallized from DMF (6.58 g, 11.8 mmol, 73.8%). mp: 375-376° C. dec; ¹H NMR (400 MHz, CDCl₃): δ/ppm=1.51 (s, 18H), 1.58 (s, 18H), 7.73 (d, J=1.0 Hz, 2H), 7.77 (d, J=1.0 Hz, 2H), 7.83 (d, J=1.0 Hz, 2H), 8.17 (d, J=1.0 Hz, 2H); ¹³C NMR (100 MHz, CDCl3): δ/ppm=31.7, 35.5, 35.6, 118.6, 120.3, 121.5, 121.6, 128.7, 130.7, 132.6, 133.9, 138.7, 144.5, 150.9, 151.4; HRMS (APPI-TOF): 556.3166 (Calcd. for C₄₀H₄₄S 556.3158).

### Example 2

In this Example, PCA-1 of Example 1 was characterized.

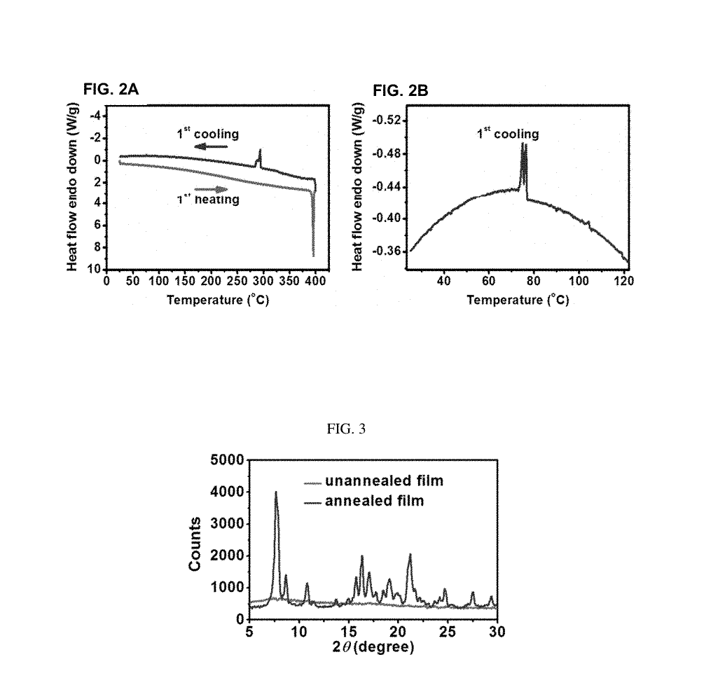

PCA-1 was characterized by differential scanning calorimetry, cyclic voltammetry, and X-ray diffraction. Differential scanning calorimetry measurements (FIG. 2) showed that PCA-1 was stable at temperature as high as 400° C. It had a melting temperature around 375° C. Thin film diffraction studies (FIG. 3) showed that PCA-1 pristine film was largely amorphous but highly crystalline after thermal annealing at 120° C. for 10 min. The sharp and strong peak at the low diffraction angle indicated excellent long-range order which was believed to be responsible for the high hole mobility described therein. The frontier molecular orbital energy levels of PCA-1 were studied using cyclic voltammetry (CV) measurements. The highest occupied molecular orbital (HOMO) and the lowest unoccupied molecular orbital (LUMO) energy levels were determined by CV to be −5.34 eV and −2.68 eV, respectively. The LUMO level (−2.68 eV) was much higher than the LUMO of MAPbI₃ (−3.9 eV), making it an excellent electron blocker. The HOMO level of PCA-1 (−5.34 eV) matched the HOMO of MAPbI₃ (−5.40 eV), facilitating hole injection from perovskite to the hole transporting PCA-1 layer.

### Example 3

In this Example, hole mobilities of PCA-1 films were determined.

The hole mobilities of PCA-a films were measured by the space-charge limited current (SCLC) method. The Hole-only devices were constructed by a spin-coated PCA-1 layer sandwiched between a PEDOT:PSS coated ITO substrate and a thermally deposited MoO₃/Au top electrode. Specifically, ITO glass substrates were etched in a specific pattern by exposure to aqua regia vapor and then cleaned in an ultrasonic bath of hot detergent, water, deionized water, toluene and isopropyl alcohol followed by UV/ozone treatment. A 45 nm thick PEDOT:PSS layer was spin-coated on the ITO substrates from an aqueous solution, and the devices were dried at 120° C. for 45 min in air. After a brief cooling, the PCA-1 solution in CHCl₃ (20 mg mL-1) was spin-coated on the PEDOT:PSS coated ITO substrates. Half of the devices were thermally annealed at 120° C. for 10 min under N₂ atmosphere in the dark. Top electrodes (10 nm thick MoO₃ and 100 nm thick Au) were thermally evaporated at <2×10⁻⁶ mbar through a shadow mask. The active area of the devices was defined to be 0.14 cm². The I-V characterization was performed in the dark using a Keithley 2400 source meter. The charge carrier (hole here) mobility was derived by fitting the J-V curves in the SCLC region using the Mott-Gurney equation:

$$J = \frac{9}{8} {ɛ}_{0} {ɛ}_{r} {μ}_{0} \frac{{( {V}_{app} - {V}_{bi} )}^{2}}{{d}^{3}}$$

where J is the current density in the SCLC region; Vₐₚₚ is the applied voltage; V_{bi} is the built-in voltage; ε₀ is the permittivity of free space (8.854×10⁻¹² F m⁻¹); εᵣ is the relative dielectric constant of the thin film; μ₀ is the zero-field charge carrier mobility; and d is the film thickness.

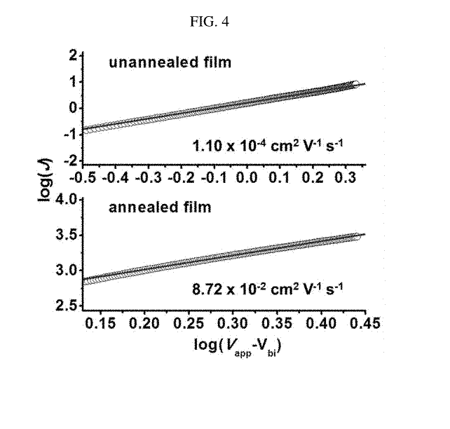

FIG. 4 shows the experimental and fitted J-V data of PCA-1 films before and after thermal annealing. The pristine thin film had a hole mobility of 1.10×10⁻⁴ cm² V⁻¹ s⁻¹. After thermal annealing (at 120° C. for 10 min), the hole mobility of the thin film was elevated to 8.72×10⁻² cm² V⁻¹ s⁻¹, which is amongst the highest reported hole mobilities obtained by SCLC method for any solution-processed small-molecule organic semiconductors. The increase of mobility by a factor of nearly three orders of magnitude for PCA-1 upon thermal annealing is attributed to its drastically increased crystallinity and long-range order as revealed by the sharp intense peaks in the XRD pattern of the thin film after thermal annealing (FIG. 3). The hole mobilities of PCA-1 films with a broad thickness range from 380 to 860 nm were measured. Consistent and reproducible high mobility values with a small standard deviation were obtained, indicating that PCA-1 film can be made in a broad range of thickness without affecting its high mobility.

2,5,9,12-tetra(tert-butyl)diacenaphtho[1,2-b:1′,2′-d]thiophene (PCA-1) was spin-coated to form thin films. The peripheral tert-butyl groups prevented π-π stacking during the spin-coating process when solvent was quickly evaporated, which resulted in a uniform and amorphous pristine film and exhibited unappealing hole mobility. After thermal annealing, the entire amorphous film became highly crystalline, yielding one of the highest space charge limited current (SCLC) hole mobilities in organic thin films measured in macroscopic device sizes.

### Example 4

In this Example, use of PCA-1 as HTL in n-i-p Planar Perovskite Solar Cells was determined.

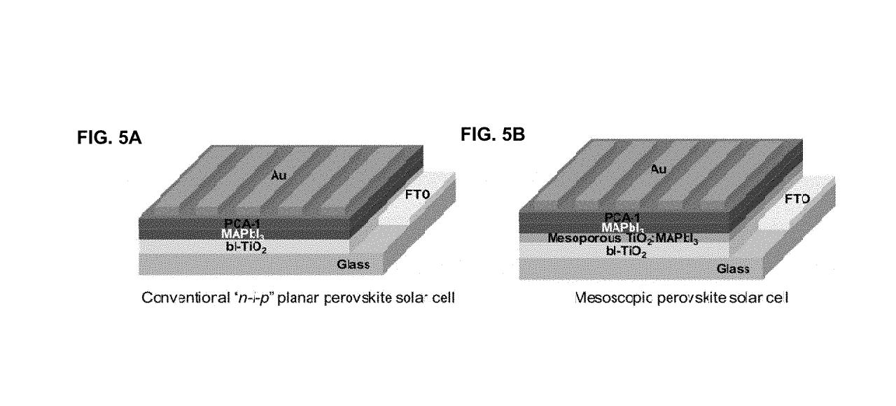

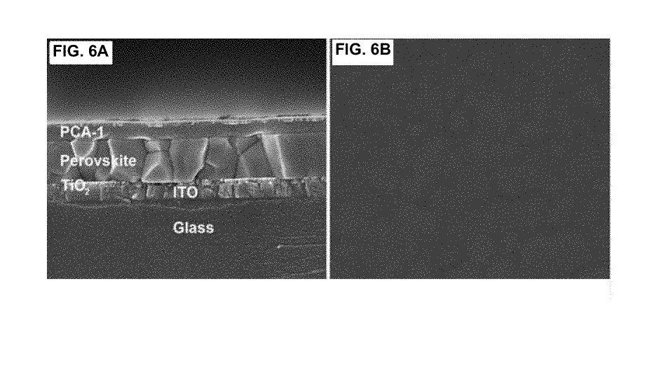

Specifically, ITO glass substrates were etched in a specific pattern by exposure to aqua regia vapor and then cleaned in an ultrasonic bath of hot detergent water, water, deionized water, toluene, acetone, and isopropyl alcohol, followed by UV/ozone treatment. A 0.1 M titanium diisopropoxide bis(acetylacetonate) solution was spin coated onto the UV/ozone-treated ITO substrates at 4000 rpm for 10 seconds and the films were annealed at 125° C. on a hotplate in air for 5 min. The substrates were subjected to a second spin-coating (5000 rpm for 10 seconds) and annealing process (125° C. on a hotplate in air for 5 minutes) using the same solution. The devices were sintered at 500° C. for 40 minutes to form a dense TiO₂ layer of ˜50 nm in thickness on top of the ITO substrates. A CH₃NH₃I.PbI₂.DMSO adduct solution was prepared by mixing 238.5 mg of CH₃NH₃I, 691.5 mg of PbI₂ and 117.2 mg of DMSO (molar ratio 1:1:1) in 900 mg of DMF at room temperature with stirring for 1 hour for a complete dissolution. The clear adduct solution was spin coated onto the TiO₂-coated ITO substrates at 4000 rpm for 25 seconds, and at ˜8 seconds after the spinning started, 0.5 mL of diethyl ether was gently dripped onto the rotating substrates within 1 second to selectively wash away the DMF. The resultant transparent CH₃NH₃I.PbI₂.DMSO adduct films were heated first at 65° C. on a hotplate in air for 1 minute and then at 100° C. on another hotplate in air for 2 minutes, and dense dark brown MAPbI₃ perovskite top layers were formed. After a brief cooling, PCA-1 solution (20 mg mL⁻¹ in chlorobenzene) was spin coated onto the perovskite layer at 1000 rpm for 40 seconds. The devices were then subjected to thermal annealing at 100° C. on a hotplate in glove box for 10 minutes. Subsequently, an 80 nm thick Au electrode was deposited on the top through a shadow mask by thermal evaporation under high vacuum (<2×10⁻⁶ mbar). The active area of the perovskite solar cells was 0.14 cm², which was defined by the overlap of ITO and Au layers. FIG. 5A depicts the device configuration.

Surface and cross-sectional FESEM images were taken with a Hitachi S-4700 field emission scanning electron microscope. As shown in FIG. 6, the PCA-1 film formed on top of the perovskite layer is uniform and pinhole-free.

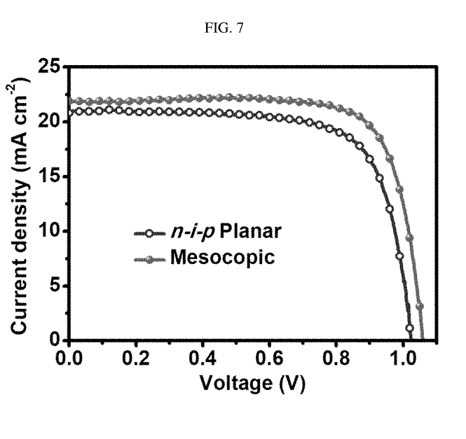

Current-voltage (I-V) responses were measured with a Keithley 2400 source meter under 1 sun AM 1.5G illumination (100 mW cm⁻²) (Oriel xenon arc lamp solar simulator). To minimize the hysteresis effect, the I-V curves were measured using reverse scan at a scan rate of 10 mV s⁻¹. FIG. 7 shows the J-V curves. The short circuit current density (J_{sc}), open-circuit voltage (V_{oc}), fill factor, and the power conversion efficiency were 20.8 mA/cm², 1.02 V, 0.73 and 15.6%, respectively. This performance is comparable if not higher than similar devices with doped spiro-OMeTAD as the HTL.

### Example 5

In this Example, use of PCA-1 as HTL in Mesostructured Perovskite Solar Cells (MSSCs) was determined.

Mesoscopic perovskite solar cells with PCA-1 (dopant-free) as the HTL were fabricated. The device structure was denoted as FTO/bl-TiO₂/mesoporous TiO₂:MAPbI₃/MAPbI₃/PCA-1/Au (FIG. 5B). Except for the two different steps mentioned below, the other fabrication and testing procedures of mesostructured perovskite solar cells were essentially the same as for the above conventional n-i-p planar perovskite solar cells.

First, FTO glass slides (instead of ITO glass slides) were used as substrates. The FTO substrates were etched using Zn dust and a 2 M HCl solution, and then cleaned in an ultrasonic bath of detergent water, acetone, isopropyl alcohol, DI water, and ethanol, followed by UV/ozone treatment. Second, after the formation of the dense bl-TiO₂ layer on FTO substrates, an additional mesoporous titanium oxide (m-TiO₂) layer was deposited by spin coating a diluted TiO₂ paste (18NR-T Transparent Titania Paste, DYESOL) (150 mg mL⁻¹ in ethanol) on the bl-TiO₂ substrates at 4000 rpm for 20 seconds. The devices were sintered at 500° C. for 30 minutes to form a mesoporous TiO₂ layer on top of the bl-TiO₂ substrates. Li ions doping of m-TiO₂ was performed by spin-coating a 0.1 M solution of lithium-bis(trifluoromethanesulfonyl)imide (Li-TFSI) in acetonitrile at 3000 rpm for 10 seconds. The devices were sintered at 450° C. for 30 minutes before the deposition of perovskite layer. The active area of the perovskite solar cells was 0.112 cm². As shown in FIG. 7, the MSSC device with PCA-1 as the hole transporting layer without any dopants gave J_{sc}, V_{oc}, fill factor, and power conversion efficiency of 21.9 mA/cm², 1.06 V, 0.77 and 17.8%, respectively. Such an efficiency is, to the inventor's knowledge, the highest among all perovskite solar cells with dopant-free HTL materials and with MAPbI₃ as the perovskite.

### Example 6

In this Example, the long term stability of perovskite solar cells with PCA-1 as the HTL in both n-i-p and MSSC device configurations was evaluated.

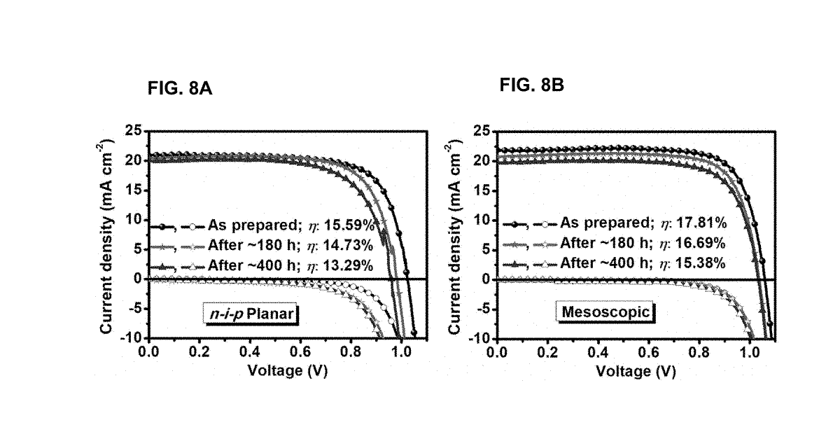

The long term stability of PSCs with PCA-1 as the HTL in both the n-i-p configuration and the MSSC architecture have been evaluated. The devices were stored at ambient conditions in dark and were measured on its performance at different time intervals. As shown in FIG. 8, the devices, whether in the n-i-p or the MSSC configuration maintained over 85% of the original efficiency after 400 hours (16 days) of storage. Under similar conditions, PSCs with doped Spiro-OMeTAD as the HTL typically lose more than 80% of its efficiency in merely 24 hours. These results unambiguously confirm that PCA-1 as the HTL significantly improves the device stability.

The present disclosure encompasses embodiments in other forms without departing from the core principles thereof. It is intended that all matter contained in the above description shall be interpreted as illustrative and not in a limiting sense.

1. A thin film composition comprising a compound of formula (I) 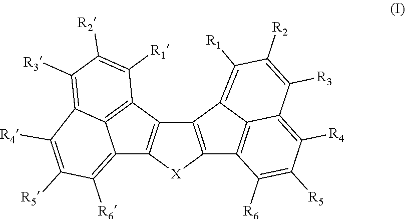
wherein X is a heteroatom of O, S, Se, and N—R′; and R₁, R₂, R₃, R₄, R₅, R₆, R₁′, R₂′, R₃′, R₄′, R₅′, R₆′, and R′, independently comprise a solubilizing group.

2. The thin film composition of claim 1, wherein the solubilizing group comprises an alkyl, a cycloalkyl, an haloalkyl, an alkenyl, an alkynyl and an arylalkyl.

3. The thin film composition of claim 2, wherein R₁, R₂, R₃, R₄, R₅, R₆, R₁′, R₂′, R₃′, R₄′, R₅′, R₆′, and R′, are independently selected from H, a C₁₋₂₀ alkyl group, a C₁₋₂₀ haloalkyl group, a C₅₋₁₄ cycloalkyl group, and a —Ar—R″ group wherein Ar is a C₆₋₁₄ aryl group and R″ is a solubilizing group.

4. The thin film of claim 1, wherein the thickness ranges from about 10 nm to about 5 μm.

5. A perovskite photovoltaic device comprising: a perovskite layer and a hole transporting layer, wherein the hole transporting layer comprises a polycyclic heteroaromatic hydrocarbon compound.

6. The perovskite photovoltaic device of claim 5, wherein the polycyclic heteroaromatic hydrocarbon compound comprises a compound of formula (I) 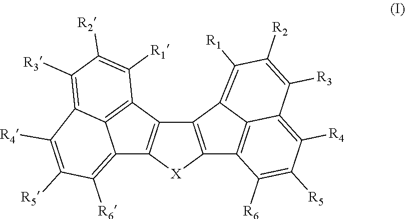
wherein X is a heteroatom of O, S, Se, and N—R′; and R₁, R₂, R₃, R₄, R₅, R₆, R₁′, R₂′, R₃′, R₄′, R₅′, R₆′, and R′, independently comprise a solubilizing group.

7. The perovskite photovoltaic device of claim 6, wherein the solubilizing group comprises an alkyl, a cycloalkyl, an haloalkyl, an alkenyl, an alkynyl and an arylalkyl.

8. The perovskite photovoltaic device of claim 6, wherein R₁, R₂, R₃, R₄, R₅, R₆, R₁′, R₂′, R₃′, R₄′, R₅′, R₆′, and R′ are independently selected from H, a C₁₋₂₀ alkyl, a C₅₋₁₄ cycloalkyl, a C₁₋₂₀ haloalkyl, a C₅₋₁₄ cycloalkyl, and a —Ar—R″, where Ar is a C₆₋₁₄ aryl and R″ is a solubilizing group.

9. The perovskite photovoltaic device of claim 6, wherein the polycyclic heteroaromatic hydrocarbon compound comprises a diacenaphtho[1,2-b:1′,2′-d]thiophene.

10. The perovskite photovoltaic device of claim 6, wherein the polycyclic heteroaromatic hydrocarbon compound is a compound of formula (II)

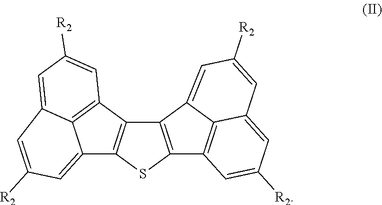

11. The perovskite photovoltaic device of claim 10, wherein each R₂ is selected from a C₁₋₂₀ alkyl group, a C₁₋₂₀ haloalkyl group, and a —Ar—R″, wherein Ar is a C₆₋₁₄ aryl group and R″ is a C₁₋₂₀ alkyl or haloalkyl group.

12. The perovskite photovoltaic device of claim 5, wherein the hole transporting material is substantially free of a dopant.

13. The perovskite photovoltaic device of claim 5, wherein the perovskite layer comprises a perovskite compound of the formula ABX₃, wherein A is selected from Cs⁺, CH₃NH₃⁺, HN═CHNH₃⁺, (CH₂)₃NH₂⁺, [CH(NH₂)₃]⁺, and combinations thereof; B is selected from Sn²⁺ and Pb²⁺; and each X independently is selected from I⁻, Br⁻, and Cl⁻.

14. The perovskite photovoltaic device of claim 5 further comprising at least one electrode.

15. The perovskite photovoltaic device of claim 5 further comprising an electron transporting layer.

16. The perovskite photovoltaic device of claim 5 comprising a n-i-p device structure.

17. The perovskite photovoltaic device of claim 5 comprising a p-i-n device structure.

18. The perovskite photovoltaic device of claim 5 further comprising at least one interfacial layer between the at least one electrode and the perovskite layer.

19. (canceled)

20. (canceled)

21. (canceled)

22. The perovskite photovoltaic device of claim 21, wherein the cathode is a C-electrode.

23. The perovskite photovoltaic device of claim 5 further comprising a mesoporous metal oxide layer.

24. (canceled)
